(19) United States
(12) Patent Application Publication
(10) Pub. No.: US 2004/0202736 A1
(43) Pub. Date: Oct. 14, 2004

(54) Method of ameliorating side-effects of SERMs

(75) Inventors: Gerardo Hernandez Munoz, Caracas (VE); Salvatore Pluchino, Colinas de Bello Monte (VE)

Correspondence Address: BOEHRINGER INGELHEIM CORPORATION, 900 RIDGEBURY ROAD, P. O. BOX 368, RIDGEFIELD, CT, 06877, US

(73) Assignee: Pharmaton S.A., Bioggio (CH)

(21) Appl. No.: 10/410,381

(22) Filed: Apr. 9, 2003

(51) Int. Cl. A61K 35/78

(52) U.S. Cl. 424/764

(57) ABSTRACT

The invention relates to a method of treatment and/or prevention of side-effect such as hot flashes caused by Selective Estrogen Receptors Modulators (SERMs) such as tamoxifen, which method comprises long term administration of an effective amount of a standardized dry extract of Cimicifuga racemosa

### BACKGROUND OF THE INVENTION

1. Technical Field

The invention relates to a method of treatment and/or prevention of side-effect such as hot flushes caused by Selective Estrogen Receptors Modulators (SERMs) such as raloxifen or tamoxifen.

2. Background Information

Aging ovaries stop secreting estradiol when menopause occurs; by then, estrone (less active estrogen) replaces estradiol. The lack of physiological feedback of estradiol production increases serum levels of both, follicle stimulating hormone (FSH) and luteinizing hormone (LH).

During this time period, quality of life is reduced in most women due to vasomotor episodes [1].

Frequent hot flushes are associated with psychosomatic complaints such as tenseness, tiredness, irritability, headaches, muscle and joint pain and depression. Vasomotor complaints vary widely in both severity and duration. Severe vasomotor complaints in menstruating women cause larger decreases in wellbeing than in non-menstruating women. As a consequence of decreased levels of estrogen, bone turnover increases and bone resorption prevails over bone formation [2]. Menopause is also linked to cardiovascular health and to reduction in cognitive function [3,4].

Postmenopausal Hormone replacement therapy (HRT) can be seen as a specific treatment for symptoms in the short term and preventive therapy in the long term. Unfortunately, data from randomized, controlled clinical trials on the impact of HRT on women's health are still lacking. Definitive answers on questions such as the possibility of increased breast cancer risk in patients on long term HRT should be available [5].

Although HRT in postmenopausal women is able to reverse several risk factors for cardiovascular disease and bone resorption, its effect on risk of breast cancer remains an unresolved issue. As would be expected, the issues of HRT become even more complicated when its use is considered for women with a known history a breast cancer. This population of postmenopausal breast cancer survivors is rising. In addition, the increasing use of adjuvant chemotherapy for patients whose axillary lymph nodes are negative or positive for disease results in an additional population of younger patients who are rendered prematurely menopausal [6]. It has been demonstrated that ovarian ablation may be associated with a 15 to 25% reduction in rates of breast cancer recurrence and of mortality [7]. This has led to speculation that the ovarian failure induced by chemotherapy may contribute to the survival benefit derived from adjuvant therapy. The evidence that estrogen has an adverse effect on breast cancer risk is based on the in vitro effects of estrogen on breast cancer cell lines and the epidemiologic data suggesting that long term use of estrogen may increase the risk of developing breast cancer.

Given the uncertainty of this situation, most clinicians choose to use alternative therapeutic routes; moreover, women with a previous diagnosis of breast cancer are averse to accepting much increased risk of recurrence in order to take HRT [8].

The Selective Estrogen Receptors Modulators (SERMs) are a new class of pharmacological agents now available to women who cannot tolerate or are unwilling to use conventional HRT. SERMs were formerly referred to as antiestrogens, a description that is now known to be inappropriate. The term SERM has been coined to describe compound that, in contrast to pure estrogen agonists or antagonists, have a mixed and selective pattern of estrogen agonist-antagonist activity, which largely depends on the tissue targeted.

The pharmacological goal of these drugs is to produce estrogenic actions in those tissues where these actions are beneficial (bone, brain, liver) and to have either no activity or antagonistic activity in tissues such as breast and endometrium, where estrogenic actions (cellular proliferation) might be deleterious. The most actively studied SERMs commonly are tamoxifen and raloxifene. Tamoxifen is currently approved as an adjuvant for the treatment of breast cancer (node-negative or node-positive) in women after total or segmental mastectomy and breast irradiation, in the treatment of woman with advanced or metastatic breast cancer, and as a preventive agent for women at high risk for breast cancer. The NSABP P-1 trial has shown that 20 mg/day of tamoxifen reduced the risk of invasive breast cancer by 49% (69 months of follow-up). The decreased risk occurred in women of all age groups with ER-positive breast cancer; it had no effect on ER-negative tumors. However, women age 50 or older receiving tamoxifen had more than a two-fold increased risk of early stage endometrial cancer; women younger than 50 had no increased incidence of adverse events, including endometrial cancer [9]. Moreover, the NSABP P-1 Study shows that tamoxifen use is associated with an increase in specific vasomotor and gynecological symptoms [10].

Hot flushes are a well known side-effect which occur in pre- and postmenopausal breast cancer patients treated with Tamoxifen. Severe hot flushes are more frequent in menstruating women with a greater impact on quality of life; the majority of them can not use conventional therapy for the control of the menopausal symptoms. Medications other than estrogen have to be used to control such symptoms among breast cancer survivors. Cimicifuga racemosa (CR) was originally used for menstrual and climateric conditions. In recent years, cimicifuga racemosa has increasingly been recommended for use in the treatment of the physical and psychological symptoms of menopause [13,14]. The exact mechanism by which cimicifuga racemosa elicits its effects on menopausal symptoms has not been elucidated. While some have proposed that its effectiveness is mediated by an inhibitory effect on the hypothalamus or an effect on neurotransmitters [15], others have suggested that it has a direct estrogenic effect with the hypothesis that cimicifuga racemosa contains phyto-estrogens, estrogen-like compounds found in plants [16]. To date, no known phyto-estrogens have been indentified in cimicifuga racemosa. An estrogenic activity is supported by some preclinical and clinical trials and not by others. The results of preclinical research addressing these hypotheses, however, are mixed.

The US patent U.S. Pat. No. 6,267,994 discloses an anti-proliferative activity of a combination of cimicifuga racemosa and tamoxifen using in vitro tests.

In a recent randomized, double masked and placebo controlled study, the effect of cimicifuga racemosa on the frequency and intensity of hot flushes was investigated in patients with history of breast cancer. The study was stratified on tamoxifen/no tamoxifen use; for eligibility women had to have completed primary therapy, including chemotherapy and radiations therapy. Both the treatment and placebo groups, whether using tamoxifen or not, reported a reduction in the intensity of hot flushes, but the difference between the treatment/no tamoxifen group and the other groups was not statistically significant [17]. A limitation of this study is that participation lasted only two months.

Accordingly, there is a high need for treating women suffering from hot flushes induced by the treatment with SERM's, to ameliorate this side-effect.

It has now be found that a long term treatment with a standardized extract of cimicifuga racemosa improves dramatically the situation of these women.

### BRIEF SUMMARY OF THE INVENTION

The invention relates to a method of treatment and/or prevention of side-effect, in particular hot flushes caused by Selective Estrogen Receptors Modulators (SERMs), which method comprising administering to a patient in need therof an effective amount of a standardized dry extract of Cimicifuga racemosa over a period of at least four months.

### DETAILED DESCRIPTION OF THE INVENTION

The term “a patient need thereof” as used hereinabove and hereinbelow relates to a healthy female person who is in need of reduction of the side-effects arising from the treatment with SERMs. As a rule such persons are adult with an age of between 30 and 60, preferably between 35 and 55 years having an mean age of about 46 years.

Preferably the standardized extract is administered to a woman after total or segmental mastectomy and breast irradiation, a woman with advanced or metastatic breast cancer, or a women at high risk for breast cancer, who is concurrently treated with an SERM, in particular to a woman of pre-menopausal status with regular menstruation and normal menstrual cycle, and breast cancer diagnosis with ER-positive tumor.

The term “effective amount” as used herein means an amount sufficient to reduce the side effects caused by the treatment of SERMs. Preferably the occurrence of hot flushes is reduced from 100% of the patients to less than 60% of the patients and the occurrence of severe flushes is reduced from about 75% of the patients to about 25% of the patients.

Under the term “extracts” are meant that the plants or plant components are extracted with a suitable solvent like water, ethanol, butanol, acetone, mixture thereof, ethers, oils or any other suitable solvent well known in the state of the art of extracting plants. These extracts can be used as such if pharmacologically acceptable or the solvent of the resulting solutions is removed and the residue is used as such or after further worked up, for example after resolving or re-suspending in a pharmacological suitable solvent.

Under the terms “active ingredients” are meant all active ingredients that are mainly responsible for the pharmacological effect. Preferably the formulation comprises all those ingredients of the plant of interest that are responsible for at least 75 percent, more preferably at least 90 percent of the pharmacological effect.

The term “pharmaceutical composition” means a composition, which is suitable for prescription and OTC medicaments, and which are available from doctors, in chemist's shop or in drugstores, only.

Preferably said SERM is raloxifen or tamoxifen, in particular wherein said patients are breast cancer survivors under adjuvant therapy of tamoxifen, which is Z-2-[4-(1,2-diphenyl-1-butenyl)phenoxy]-N,N-dimethyl-ethylamine.

Most preferably 10 to 30, in particular about 20 mg/day of tamoxifen are administered to said patient.

Preferably the standardized dry extract of Cimicifuga racemosa is administered to the patient for at least six months, in particular for six to sixty, most preferably six to twenty four months.

Preferably 10 to 30, in particular 20 mg of said standardized dry extract of Cimicifuga racemosa are administered to the patient twice a day.

Under the term “plant” is understood the plant itself as well as plant parts comprising the active ingredients. Like for example the leaves, the stems, the seeds, the fruits, rhizomes or roots as mentioned above. Preferably the plant or plant components are dried. Optionally, they may be cut to pieces, ground or powdered.

Under the term “extracts” are meant that the plants or plant components are extracted with a suitable solvent like water, ethanol, butanol, acetone, mixture thereof, ethers, super critical carbon dioxide, halogenated hydrocarbons such as dichloromethane, oils or any other suitable solvent well known in the state of the art of extracting plants. These extracts can be used as such if pharmacologically acceptable or the solvent of the resulting solutions is removed and the residue is used as such or after further worked up, for example after resolving or re-suspending in a pharmacological suitable solvent.

Preferably the standardized dry extract of Cimicifuga racemosa is obtained by extraction of plant parts, in particular the rhizome of black cohosh with an organic solvent, most preferably with aqueous ethanol, or super-critical carbon dioxide and the resulting “wet extract” is concentrated and dried as disclosed by the International patent application WO 99/47149, of which the complete disclosure is herewith incorporated by reference.

Preferably the standardized dry extract of Cimicifuga racemosa is administered in the form of a capsule or tablet, in particular a film coated tablet, most preferably in form of the commercially available BNO 1055 (MENOFEM®) from Boehringer Ingelheim-Pharmaton.

The SERM and the standardized dry extract of Cimicifuga racemosa may be administered subsequently in different dosage forms or together in one single combined dosage form.

It is particularly preferred to administer both active ingredients separately. For the sake of patients' compliance the SERM and the standardized dry extract of Cimicifuga racemosa are preferably provided in the form of a pharmaceutical kit with two compartments one of which (A) comprising the SERM and the other comprising said extract of Cimicifuga racemosa.

Accordingly the invention relates to a pharmaceutical kit comprising at least two separate unit dosage forms (A) and (B):

(A) one of which comprises a composition containing tamoxifen and a pharmaceutically acceptable carrier, in particular a tablet containing 10 to 30, most preferably about 20 mg of tamoxifen;

(B) one of which comprises a composition containing a standardized dry extract of Cimicifuga racemosa and a pharmaceutically acceptable carrier, in particular two tablets containing each 10 to 30, most preferably about 20 mg of a standardized dry extract of Cimicifuga racemosa.

In another preferred embodiment the standardized extract of Cimicifuga racemosa is administered simultaneously with the SERM, preferably in the form of a coated tablet, in which the core consists essentially of a standardized dry extract of Cimicifuga racemosa embedded in a coating layer comprising the SERM, in particular tamoxifen and optionally at least one component selected from the group consisting of calcium salts, binders, disintegrants, wetting agents, fillers, lubricants and colorants. Similar coated tablet comprising cimicifuga and calcium salts are disclosed by the International patent application WO 02/100422 A1, the complete disclosure of which, in particular the examples and drawings thereof, are herewith incorporated by reference.

The following described clinical trial has been carried out to assess the efficacy and side effects of CR extract BNO 1055 in controlling hot flushes among breast cancer survivors under tamoxifen adjuvant therapy.

Methods

The Clinical Practice

At the Mastology Unit of the Centro Clinico de Maternidad “Leopoldo Aguerrevere”, 1,600 breast cancer survivors women of all types, irrespective of age, menopausal, nodal and ER status have been treated with tamoxifen (20 mg/day) (table 1).

TABLE 1
TO WHOM TAMOXIFEN WAS OFFERERD

```
Women 35-50 with a 5-years Gail risk ≧1.7%
Women >50
  Without a uterus
  With a uterus
Women with a history of
  ER-positive ductal carcinoma
  ER-positive lobular carcinoma
  In situ or infiltrating carcinoma
  ER-negative tumor
```

Tamoxifen duration therapy for the patients is usually programmed for a five years period. The improvements in recurrence-free survival and in overall survival have been between the ranges reported by the published literature. However, into the total treated group, we have found only one case of endometrial cancer, well differentiated and of small size (table 2).

TABLE 2
ADVERSE EFFECTS OF TAMOXIFEN

Menopausal symptoms
Hot flushes
Psychosomatic complaints
Irregular menses
Ocular toxicity
Gynecologic complications
Vaginal discharge
Endometrial hyperplasia and polyps
Ovarian cysts
Endometrial cancer (low grade)

Tamoxifen has been extremely well tolerated by the patients and withdrawal has been fewer than 5%. The most common adverse effects of tamoxifen have been vasomotor symptoms, being more common in women before menopause than afterward: 25% of postmenopausal patients reported hot flushes, while in premenopausal women hot flushes were higher up to 74%, mostly severe. Many of these women had recently stopped HRT, at the time the diagnosis of breast cancer was made. In order to improve the quality of life of patients treated with tamoxifen, in separate experiences we tried to control vasomotor symptoms by the use of clonidine, high dose of Vitamin E or progesterone, but with very unsatisfactory results. On the contrary, very promising results were obtained when tamoxifen was administered together with the standardized extract of CR, BNO 1055.

Women with breast cancer are at higher risk for problems from hot flushes for several reasons. When menopause occurs abruptly, as in the case of surgical oophorectomy or with alkylating chemotherapy, symptoms can be more severe, and tamoxifen, of course, can induce hot flushes.

Most women who undergo spontaneous menopause may not seek treatment for hot flushes. However, when these symptoms are severe, they can have a significantly adverse effect on quality of life. Since the number of breast cancer survivors is expected to increase, this clinical problem is expected to become more common.

Study recruitment begun in May 1999. The main reason for eligibility was the pre-menopausal status with regular menstruation and normal menstrual cycle, and breast cancer diagnosis with ER-positive tumor. The most common reason for ineligibility was refusal to consider a study treatment for relief of symptoms, a history of other cancers and serious chronic medical conditions. Ultimately, 90 women were included into the study; on average, these women where 46 years old (35 to 52 years). To 10% of them a total hysterectomy with retention of the adnexa had been performed (Table 3).

TABLE 3
MEDICAL AND DEMOGRAPHIC CHARACTERISTICS
OF 136 FEMALE BREAST CANCER SURVIVORS

| | Usual-Care Group | Intervention Group |
|---|---|---|
| Characteristic | n = 46 | n = 90 |
| Age | 47 (36-51) | 46 (35-52) |
| Ethnicity | | |
| Hispanic | 30 (65%) | 52 (57%) |
| White | 16 (35%) | 38 (43%) |
| Type of Surgery | | |
| Lumpectomy | 28 (60%) | 59 (65%) |
| Mastectomy | 18 (40%) | 31 (35%) |
| Cancer Treatment | | |
| Tamoxifen | 46 (100%) | 90 (100%) |
| Prior Radiation Therapy | 27 (60%) | 65 (72%) |
| Prior Chemotherapy | 20 (45%) | 42 (47%) |
| Node | | |
| Negative | 29 (62%) | 59 (65%) |
| Positive | 17 (38%) | 31 (35%) |
| Medical Conditions | | |
| Arthritis | 12 (25%) | 25 (28%) |
| Hypertension | 6 (12%) | 14 (15%) |
| Other | 8 (17%) | 12 (13%) |

Physical and gynecologic examination with Pap smear were performed, together with intravaginal ultrasound which was repeated 6 months later and successively every year, in order to measure the endometrium thickness; in those cases were the endometrial hypertrophy was grater than 10 mm biopsy was taken. Patients under therapy were evaluated every two months.

Tumor stages T1, T2, T3 (according to the TNM classification of the IUAC) were diagnosed and treated: T1 and T2 (tumor up to 3 cm) received lumpectomy, followed by radiation therapy and, when node was positive, adjuvant chemotherapy. Clinically and surgically, 65% of patients were node negative, while 20% had 1 to 3 positive nodes and 15% had 4 or more positive nodes. Women with tumor T2>4 cm or T3 (5 to 10 cm) were treated with chemotherapy pre- and post-mastectomy and radiation therapy after surgery. After chemotherapy and radiation therapy were completed, the patient was ready to start tamoxifen therapy (20 mg/day) for a period of five years which corresponds to the usual program applied in our Unit fifteen days before the initiation of the therapy with tamoxifen, we prescribed 20 mg, twice a day, of the standardized extract of CR, BNO 1055, which was administered with tamoxifen as a combined therapy for periods of six to twelve months until the vasomotor episodes were controlled.

Results

Of the 150 patients who initially enrolled and completed the menopausal symptoms index, 14 decided not to participate in the rest of the study, although they maintained the tamoxifen adjuvant therapy. The participants into the study were 136, of whom 90 were treated with the combination therapy tamoxifen-CR extract BNO 1055, while 46 were randomly assigned to the usual-care group. The medical and demographic characteristics of the study participants are comparable between the two groups (Table 3). To 10% of them a total hysterectomy with retention of the adnexa had been performed. All those who remained into the study (136) completed hot flushes diaries at baseline, at every control visit and at the end of the study.

Among the 46 study participants included into the usual-care group, 73.9% suffered severe hot flushes and 26.1% moderate symptoms. Patients included in this group were maintained into the study during six months without any therapy for hot flushes.

Afterward, many of the women randomized to usual-care group took open-label therapy for hot flushes prescribed by their personal physicians. In the course of the study, the bimonthly reported numbers of hot flushes in the usual-care group showed some decline from baseline. However, the difference between values at baseline and at six-months of initiating tamoxifen adjuvant therapy were not significant either for severe or for moderate hot flushes (5 to 9% decline; P=0.71; stratified Wilcoxon test from baseline to completion difference). Among the 90 study participants included into the intervention group, at the end of the study 46.7% were free of hot flushes, while only 24.4% still suffered severe symptoms (Table 4).

TABLE 4
HOT FLUSHES REDUCTION BY BNO 1055 (CR)

| | Usual-Care Group* | Intervention Group** |
|---|---|---|
| Hot Flushes | n = 46 | n = 90 |
| Severe | 34 (73.9%) | 22 (24.4%) |
| Moderate | 12 (26.1%) | 26 (28.9%) |
| None | — | 42 (46.7%) |

*Tamoxifen adjuvant Therapy
**Combined Therapy: Tamoxifen + BNO 1055 (CR)

The patterns were significantly different between the two groups on testing the differences between proportions with Fisher's exact test (P<0.01).

Eleven minor adverse events occurred: seven in the usual-care group and four in the intervention group. No serious events were reported.

Discussion

The SERM tamoxifen is first-line therapy for the hormonal treatment of breast cancer, both for adjuvant treatment and as a preventive therapy for women at high risk for breast cancer. The most frequent adverse reaction to tamoxifen include hot flushes. Most women who undergo spontaneous menopause may not seek treatment for hot flushes. However, when these symptoms are severe, they can have a significant adverse effect on quality of life. Since the number of breast cancer survivors is expected to increase, this clinical problem is also likely to become more common.

Alternative menopause therapies have to be sought for those patients who have contraindications to HRT or who reject a hormonal based regime: this is the case of breast cancer survivors women. Surgically or pharmacologically induced hormonal deficiency causes severe vasomotor episodes which adversely affect the quality of life of many women to a greater or lesser degree.

The rhizome of CR has been traditionally used in the treatment of menopausal symptoms with good results. Particularly, the extracts from the rhizome of CR show convincing effects in various estrogen-deficiency states.

In the intervention group, the previous administration of the standardized CR extract BNO 1055 greatly reduced the vasomotor episodes produced by tamoxifen, and eventually, by chemotherapy in breast cancer survivors women. Uncontrolled, comparative study with clonidine, high dose of Vitamin E or progesterone failed to reach a satisfactory control of the severe hot flushes experienced by premenopausal breast cancer survivors treated with tamoxifen.

When used for a longer period of time, cimicifuga racemosa may show greater efficacy relative to placebo. In accordance with this statement, we have demonstrated that combination therapy tamoxifen and CR extract BNO 1055, extract administered for a longer period of time, represents a very important therapeutic tool for the reduction of hot flashes and an improvement of the quality of life in this group of patients. A similar combined therapy may be used when tamoxifen is administered as a preventive agent for women at high risk for breast cancer.

Phyto-estrogens are currently a popular method of treating hot flushes for many women, and breast cancer survivors are among this group. These agents have enormous appeal because they are “natural” and because they are claimed to be safe.

If women choose to use phyto-estrogens, physicians should recommend that they should use only products that detail all of theirs ingredients and contain standardized extracts.

### REFERENCES

[1] Daly E, Gray A, Barlow D et al. Measuring the impact of menopausal symptoms in quality of life. Br Med J 1993; 307: 836-40.

[2] Riggs B I, Melton L J. The prevention and treatment of osteoporosis. N Engl J Med 1992; 327: 620-7.

[3] Colditz G A, Willet We, Stampler M J et al. Menopause and the risk of coronary heart disease in women. N Engl J Med 1987; 316: 1405-10.

[4] Paganini-Hill A, Henderson V W. Estrogen deficiency and risk of Alzheimer's disease in woman. Am J Epidem 1997; 140: 256-65.

[5] Writing Group for the Women's Health Initiative Investigators. Risks and benefits of estrogen plus progestin in healthy postmenopausal women. JAMA 2002; 288(3):321-33.

[6] Hulley S, Furberg C, Barrett-Connor E et al, for the HERS Research Group. Noncardiovascular Disease Outcomes During 6.8 years of Hormone Therapy. Heart and estrogen/progestin replacement study follow-up (HERS II). JAMA 2002;288(1):58-66.

[7] Dnistrian A M, Schwartz M K, Fracchia A A et al. Endocrine consequences of CMF adjuvant therapy in premenopausal and postmenopausal breast cancer patients. Cancer 1983; 51: 803-7.

[8] Early Breast Cancer Trialists Collaborative Group. Ovarian ablation in early breast cancer: Overview of the randomized trials. Lancet 1996; 348: 1189-96.

[9] Ganz P A, Greendale G, Kahn B, O'leary J F. Are breast cancer survivors willing to take hormone replacement therapy? Proc Am Soc Clin Oncol 1996;15: 102-6.

[10] Walsh B W, Kuller L H, Wild R A, et al. Effects of raloxifene on serum lipids and coagulation factors in healthy postmenopausal women. JAMA 1998;279(18): 1445-51.

[11] Fisher B et al. Tamoxifen for prevention of breast cancer: report of the National Surgical Adjuvant Breast and Bowel Project P-1 Study J Natl Cancer Inst 1998; 90: 1371-88.

[12] Day R, Ganz P A, Constantino J P, Cronin W M, Wickerham D L, Fisher B. et al. Health-related quality of life and tamoxifen in breast cancer prevention: a report from the National Surgical Adjuvant Breast and Bowel Project P-1 study. J Clin Oncol 1999; 17: 2659-69.

[13] Stoll W. Phytotherapeutikum beeinflusst atrophisches vaginalepitel. Therapeutikon 1987; 1:23-31.

[14] Liske E, Boblitz N, Henneicke-von Zepelin H H. Therapy of climateric complaints with cimicifuga racemosa; data on effect and efficacy from a randomized controlled double blind study. In: Rietbrock N, Donath M F, Loew D, Roots I, Schulz V, eds. Phitopharmaka V I. Darmstadt: Steinkopff, 2000;247-57.

[15] Einer-Jensen N, Zhao J, Andersen K P, Kristoffersen K. Cimicifuga and Melbrosia lack oestrogenic effects in mice and rats. Maturitas 1996;25: 149-53.

[16] Kruse S O, Löhning A, Pauli G F, Winterhoff H, Nahrstedt A. Fukiic and piscidic acid esters from the rhizome of Cimicifuga racemosa and the in vitro estrogenic activity of fukinolic acid. Planta Med 1999:65(8):763-4.

[17] Jacobson J S, Troxel A B, Evans J et al. Randomized trial of Black Cohosh for the treatment of hot flashes among women with a history of breast cancer. J Clin Oncol 2001; 19(10):2739-45.

We claim:

1. A method of treatment and/or prevention of side-effects caused by Selective Estrogen Receptors Modulators (SERMs), said method comprising administering to a patient in need thereof an effective amount of a standardized dry extract of Cimicifuga racemosa over a period of at least four months.

2. The method according to claim 1, wherein said patient selected from the list consisting of a woman after total or segmental mastectomy and breast irradiation, a woman with advanced or metastatic breast cancer, or a women at high risk for breast cancer.

3. The method according to claim 2, wherein said patient is a woman of pre-menopausal status with regular menstruation and normal menstrual cycle, and breast cancer diagnosis with ER-positive tumor.

4. The method according to claim 1, wherein said SERM is raloxifen or tamoxifen.

5. The method according to claim 4, wherein 10 to 30 mg/day of tamoxifen are administered to said patient.

6. The method according to claim 4, wherein said patients are breast cancer survivors under tamoxifen adjuvant therapy.

7. The method according to claim 1, wherein said standardized dry extract of Cimicifuga racemosa is administered to the patient for at least six months.

8. The method according to claim 7, wherein 10 to 30 mg of said standardized dry extract of Cimicifuga racemosa is administered to the patient twice a day.

9. The method according to claim 1, wherein said side effects are hot flushes.

10. The method according to claim 9, wherein said side effects are hot flushes associated with psychosomatic complaints such as tenseness, tiredness, irritability, headaches, muscle and joint pain and depression.

11. The method according to claim 8, wherein said standardized dry extract of Cimicifuga racemosa is obtained by extraction of plant parts of cimicifuga racemosa with an organic solvent or super-critical carbon dioxide.

12. The method according to claim 11, wherein said standardized dry extract of Cimicifuga racemosa is administered in the form of a tablet.

13. The method according to claim 12, wherein said standardized dry extract of Cimicifuga racemosa is commercially available as MENOFEM® from Boehringer Ingelheim-Pharmaton.

14. A pharmaceutical kit comprising at least two separate unit dosage forms (A) and (B):
(C) one of which comprises a composition containing tamoxifen and a pharmaceutically acceptable carrier;
(D) one of which comprises a composition containing a standardized dry extract of Cimicifuga racemosa and a pharmaceutically acceptable carrier.

15. A pharmaceutical kit according to claim 14, wherein
(A) comprises a tablet containing 10 to 30 mg of tamoxifen;
(B) comprises two tablets containing each 10 to 30 mg of a standardized dry extract of Cimicifuga racemosa.

16. A coated tablet, in which the core consists essentially of a standardized dry extract of Cimicifuga racemosa, which is embedded in a coating layer comprising the SERM, and at least one component selected from the group consisting of calcium salts, binders, disintegrants, wetting agents, fillers, lubricants and colorants.
